(19) United States
(12) Patent Application Publication
(10) Pub. No.: US 2024/0307268 A1
(43) Pub. Date: Sep. 19, 2024

(54) MEDICINE DISPENSING SYSTEM AND A METHOD FOR FACILITATING MEDICINE DISPENSING

(71) Applicant: Logistics and Supply Chain MultiTech R&D Centre Limited

(72) Inventors: Chi Hung TONG, HK (HK); Ka Lun FAN, HK (HK); Chung Hang LEUNG, HK (HK); Lok POON, HK (HK); Chi Hung CHUNG, HK (HK); Man Kit WONG, HK (HK); An Ling LIU, HK (HK); King Loong TSE, HK (HK); Yuk Yu LAU, HK (HK); Bik Shan LAM, HK (HK)

(21) Appl. No.: 18/184,154

(22) Filed: Mar. 15, 2023

(51) Int. Cl. A61J 7/00; G16H 20/13

(52) U.S. Cl. CPC A61J 7/0084; G16H 20/13; A61J 2200/74; A61J 2205/00

(57) ABSTRACT

A medicine dispensing system and a method for facilitating medicine dispensing. The system comprises a dispenser control module arranged to receive a medication prescription including at least one prescription item; a cabinet control module arranged to locate a position of each of the at least one prescription item stored in one of a plurality of predetermined compartment of a cabinet; and an indicator module arranged to provide a visual indication to a dispenser indicating a physical location of each of the at least one prescription item.

### TECHNICAL FIELD

The invention relates to a medicine dispensing system and a method for facilitating medicine dispensing, and particularly, although not exclusively, to a medicine dispensing system facilitating dispensing of traditional Chinese medicine.

### BACKGROUND

A medication prescription is a written order from a healthcare provider to a pharmacist or other healthcare professional to dispense a medication to a patient. The prescription typically includes the name of the medication, the dosage and frequency of administration, and any special instructions or precautions.

Once a medication prescription is received, a pharmacist or other healthcare professional will dispense the medication to the patient. Depending on the medication, the medication may be dispensed in a pill or tablet form, a liquid form, or a topical form such as a cream or ointment. The medication may be dispensed in a single dose or in a multi-dose container such as a bottle or blister pack.

The patient will typically be given instructions on how to take the medication, including the dosage, frequency of administration, and any special instructions or precautions. It is important for patients to follow these instructions carefully to ensure that the medication is effective and to avoid any potential side effects or adverse reactions.

### SUMMARY OF THE INVENTION

In accordance with a first aspect of the present invention, there is provided a medicine dispensing system comprising: a dispenser control module arranged to receive a medication prescription including at least one prescription item; a cabinet control module arranged to locate a position of each of the at least one prescription item stored in one of a plurality of predetermined compartment of a cabinet; and an indicator module arranged to provide a visual indication to a dispenser indicating a physical location of each of the at least one prescription item.

In accordance with the first aspect, the system further comprises an electronic weight arranged to weigh the prescription item according to the medication prescription.

In accordance with the first aspect, the system further comprises an optical imager arranged to identify the prescription item being weighed.

In accordance with the first aspect, the system further comprises a dispenser input module arranged to receive a manual confirmation indicating that the prescription item has been weighed and dispensed.

In accordance with the first aspect, the dispenser control module is further arranged to update the visual indication provided to the dispenser indicating the physical locations of prescription items which have not been weighed and dispensed.

In accordance with the first aspect, the indicator module comprises a plurality of indicators each provided on a compartment of the cabinet, and wherein the physical locations of prescription items which have not been weighed and dispensed are indicated by lighting up an indicator provided on the respective compartment of the cabinet.

In accordance with the first aspect, the system further comprises a display module arranged to notify the dispenser an error or a potential error associated with the at least one prescription item of the medication prescription.

In accordance with the first aspect, the error includes the at least one prescription item being overweighed or incorrected dispensed.

In accordance with the first aspect, the potential error includes a wrong prescription item being selected due to confusion.

In accordance with the first aspect, the system further comprises a confusion matrix module arranged to generate the visual indication to the dispenser indicating physical locations of a plurality of confusing prescription items in a staggered manner.

In accordance with a second aspect of the present invention, there is provided a method for facilitating medicine dispensing, comprising the step of: receiving a medication prescription including at least one prescription item; locating a position of each of the at least one prescription item stored in one of a plurality of predetermined compartment of a cabinet; and providing a visual indication to a dispenser indicating a physical location of each of the at least one prescription item.

In accordance with the second aspect, the method further comprises the step of weighing the prescription item according to the medication prescription.

In accordance with the second aspect, the method further comprises the step of identifying, by an optical imager, the prescription item being weighed.

In accordance with the second aspect, the method further comprises the step of receiving a manual confirmation indicating that the prescription item has been weighed and dispensed.

In accordance with the second aspect, the method further comprises the steps of updating the visual indication provided to the dispenser indicating the physical locations of prescription items which have not been weighed and dispensed.

In accordance with the second aspect, the physical locations of prescription items which have not been weighed and dispensed are indicated by lighting up an indicated provided on the respective compartment of the cabinet.

In accordance with the second aspect, the method further comprises the step of notifying the dispenser an error or a potential error associated with the at least one prescription item of the medication prescription.

In accordance with the second aspect, the error includes the at least one prescription item being overweighed or incorrected dispensed.

In accordance with the second aspect, the potential error includes a wrong prescription item being selected due to confusion.

In accordance with the second aspect, the method further comprises the step of providing the visual indication to the dispenser indicating physical locations of a plurality of confusing prescription items in a staggered manner.

### BRIEF DESCRIPTION OF THE DRAWINGS

Embodiments of the present invention will now be described, by way of example, with reference to the accompanying drawings in which:

FIG. 1 is a block diagram of a medicine dispensing system in accordance with an embodiment of the present invention.

FIG. 2 is an image showing a prescription being prepared and provided to the medicine dispensing system in FIG. 1 for further process.

FIG. 3 is an image showing the medicine dispensing system of FIG. 1.

FIG. 4 is an illustration showing a screenshot of a display module of the medicine dispensing system with a prescription being selected and the indicator module deployed on the medicine cabinet in FIG. 3 with LEDs lighting up showing positions of the prescription items to be withdrawn.

FIG. 5 is an illustration showing a screenshot of the display module FIG. 4 with another prescription being selected and the indicator module deployed on the medicine cabinet in FIG. 3 with another sets of LEDs lighting up showing positions of the prescription items to be withdrawn.

FIG. 6 is a flow diagram showing an example operation of the medicine dispensing system in accordance with an embodiment of the present invention.

FIG. 7 is an illustration showing an image of a first prescription item of the prescription in FIG. 5 being weighed, and real-time screenshots of the user tablet and the control module of the medicine dispensing system in accordance with an embodiment of the present invention.

FIG. 8 is an illustration showing an image of the medicine dispensing system of FIG. 7 after a second prescription item of the prescription in FIG. 5 has been weighed, and real-time screenshots of the user tablet and the control module of the medicine dispensing system.

FIG. 9 are images showing the LEDs on the drawer of the cabinet of FIG. 7 being deactivated after receiving a manual confirmation from the user that the second prescription item has been withdrawn and verified.

FIG. 10 is an illustration showing an image of all three prescription items of the prescription in FIG. 5 being weighed and dispensed, and real-time screenshots of the user tablet and the control module of the medicine dispensing system of FIG. 7.

### DETAILED DESCRIPTION OF THE PREFERRED EMBODIMENTS

The inventors devised that, for western medicine, there are many different types of medications available, each with its own unique properties and uses. These medications can be dispensed in a variety of forms, including tablets, capsules, liquids, and injections.

The process of dispensing western medicine typically begins with pharmaceutical companies that manufacture and distribute the medications to pharmacies. These pharmacies then dispense the medications to patients who have a prescription from a licensed healthcare provider.

The healthcare provider determines the appropriate medication, dosage, and frequency based on the patient's medical condition, age, weight, and other factors. Once the prescription is written, the patient takes it to the pharmacy where a licensed pharmacist reviews the prescription and prepares the medication for the patient.

The pharmacist provides the medication to the patient along with instructions on how to take it and any potential side effects or interactions to look out for. In some cases, the pharmacist may also provide additional counseling or support to the patient, such as advice on managing side effects or instructions for storing the medication.

Different from Western medicine, traditional Chinese Medicine includes herbs such as ginseng, ginger, licorice root, and astragalus. Additionally, Traditional Chinese Medicine may incorporate animal products such as dried seahorses, deer antlers, and turtle shells, although the use of these products is controversial and not widely practiced.

Traditional Chinese Medicine prescription is dispensed in various forms such as herbal decoctions, powders, pills, capsules, and tinctures. The specific form of the prescription depends on the patient's unique condition and the preference of the practitioner. In general, the prescription is formulated based on the principles of Traditional Chinese Medicine, which involve identifying imbalances and treating them with a combination of herbs and other natural substances. The practitioner may also recommend dietary and lifestyle changes to support the healing process.

After the prescription is formulated, it is typically dispensed in one of two ways. The first is through a traditional herbal pharmacy, where the patient can pick up the raw herbs and prepare the decoction at home. This method allows for a highly customized approach, as the practitioner can tailor the dosage and combination of herbs to the patient's specific needs. However, it can also be time-consuming and difficult for some patients to prepare the decoction correctly.

The second method is through pre-prepared herbal products, such as powders, pills, and capsules. These products are more convenient to use, as they are pre-measured and ready to take. However, they may not be as tailored to the patient's individual needs, as they typically contain a standard combination of herbs.

It is important to note that Traditional Chinese Medicine prescriptions should always be dispensed by a qualified practitioner who has undergone extensive training in the principles and practice of Traditional Chinese Medicine.

Chinese herbalists have a highly specialized approach that involves the careful selection and combination of specific herbs to create a customized formula for each patient. The process of preparing a prescription can be complex and time-consuming, as the herbalist must consider multiple factors such as the patient's symptoms, medical history, and overall constitution.

After assessing the patient, the herbalist will choose herbs that are appropriate for the patient's specific needs and diagnosis. Each herb has a unique set of properties and functions, and the herbalist will select herbs based on their individual properties and how they interact with other herbs in the formula.

Once the herbs have been selected, the herbalist will determine the appropriate dosage and preparation method. Some herbs may be boiled in water to create a decoction, while others may be ground into a powder or made into pills or tinctures. The preparation method will depend on the specific herbs being used and the intended effect of the formula.

After the prescription has been prepared, the patient will typically take the herbs as directed by the herbalist. The herbalist will monitor the patient's progress over time and may adjust the formula as needed based on the patient's response to the treatment.

Chinese herbal medicine has a long history and profound cultural background in China, and is widely used in traditional Chinese medicine diagnosis, treatment, and healthcare. Chinese herbal medicine can regulate the internal environment of the body, promote the balance and recovery of functions, and has unique advantages in relieving symptoms and improving immune function. However, the use of Chinese herbal medicine also has its risks, and improper use may lead to some side effects and adverse reactions.

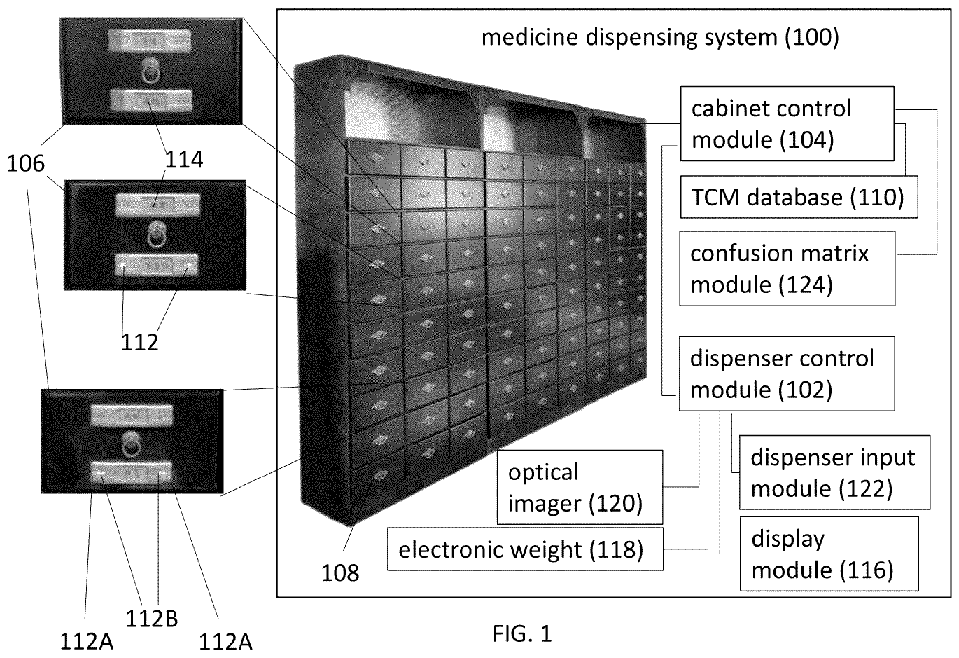

With reference to FIG. 1, there is shown an embodiment of a medicine dispensing system 100 comprising: a dispenser control module 102 arranged to receive a medication prescription including at least one prescription item; a cabinet control module 104 arranged to locate a position of each of the at least one prescription item stored in one of a plurality of predetermined compartment 106 of a cabinet 108; and an indicator module arranged to provide a visual indication to a dispenser indicating a physical location of each of the at least one prescription item.

In this example embodiment, the medicine dispensing system 100 may help can help a pharmacist or herbalist by reducing the time and effort required to dispense medication to patients. The medicine dispensing system 100 may be programmed to accurately locate the correct medication or prescription stored in a medicine cabinet 108, which may comprise a plurality of drawers or compartments 106 storing different prescription items. Advantageously, the dispenser can help reduce errors in medication dispensing, which can be particularly important for patients who are taking multiple medications or have complex medication regimens.

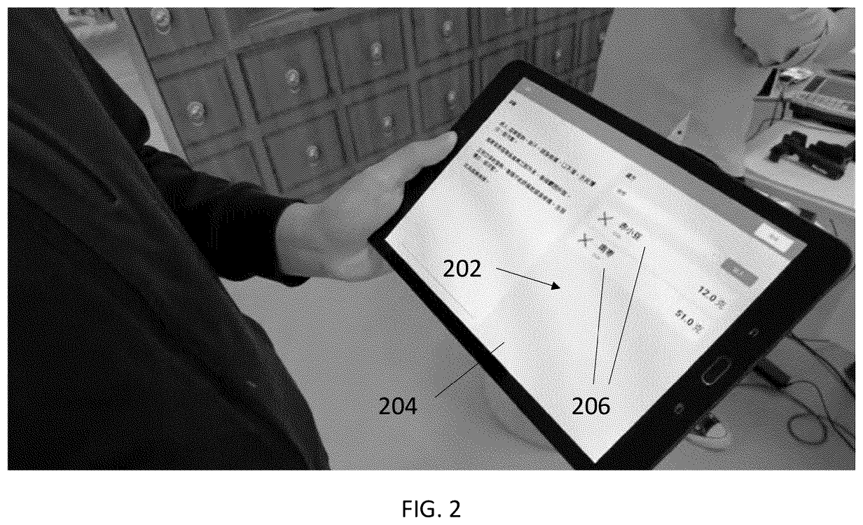

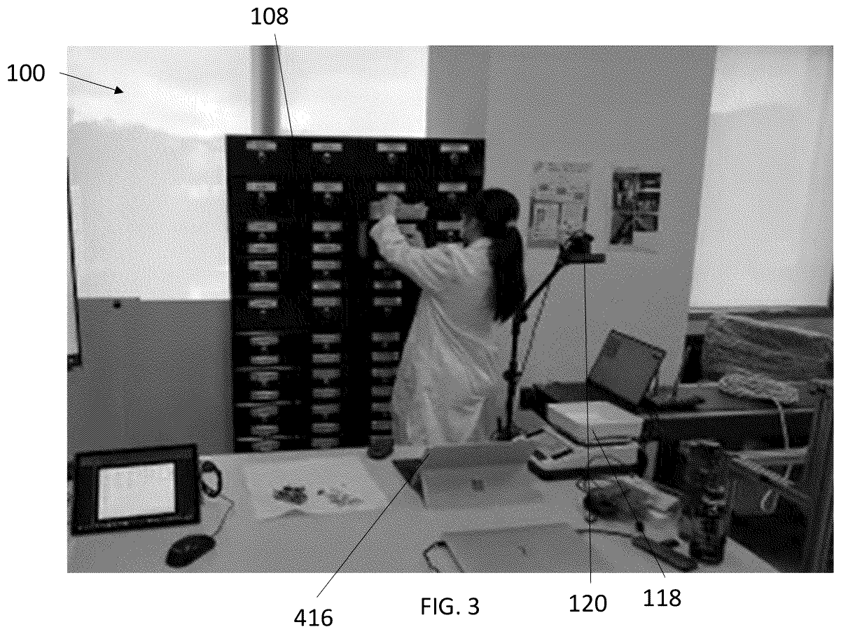

Referring to also to FIGS. 2 and 3, the medicine dispensing system 100 comprises a dispenser control module 102 arranged to receive a medication prescription including at least one prescription item. The dispenser control module 102 may coordinate all different modules in the medicine dispensing system 100 in response to different inputs or predetermined events, so as to control these different modules to provide suitable output to assist in a medicine dispensing process.

The functional units and modules of the medicine dispensing system 100 in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

Referring to FIG. 2, the medication prescription 202 may be prepared using a tablet computer 204, in which an herbalist may select the prescription items 206 stored in a database, such as a traditional Chinese medicine (TCM) database 110, and mark the appropriate weights of each of the selected prescription items. Once the preparation of the medication prescription is completed, the medication prescription may be assigned with an ID and sent to the dispenser control module 102 for further process.

For example, a medication prescription 202 including two prescription items, i.e. rice bean (also known as chixiaodou) and south dates (also known as nanzao), may be provided to the medicine dispensing system 100. In addition, each of the prescription items 206 should also include a weight assigned according to the effect of the corresponding prescription item 206. In this example, the medication prescription includes 12.0 g of rice bean and 51.0 g of south dates.

Referring to FIG. 3, the medicine dispensing system 100 further comprises other modules and/or stations arranged to facilitate dispensing of medication, such as traditional Chinese medicine prescriptions. Preferably, the system 100 comprises a cabinet control module 104 which may be used to locate a position of each of the at least one prescription item stored in one of a plurality of predetermined compartment 106 of a cabinet 108. In this example. The cabinet 108 may be a TCM cabinet (also known as baizigui), which is a type of traditional Chinese medicine cabinet with many small drawers for storing herbal remedies and other medicinal ingredients. It may be used by pharmacists and doctors to store and organize their supplies.

Preferably, the cabinet control module 104 may be deployed on top of the TCM cabinet 108, so as to fully utilize space available between the top of the cabinet and the ceiling. For example, a room may be defined with a height of 4 meters from the floor surface to the ceiling, and top row of the TCM cabinet may be of around 2 meters, i.e.

maximum height that may be reachable by a medicine dispenser, thus the space that above the TCM cabinet may be used for the deployment of the cabinet control module 104. In one example embodiment, an ASRS (i.e. automated storage and retrieval systems) or mini-ASRS control module may be used as the cabinet control module 104, to facilitate controlling different functional components in the cabinet 108, where the mini ASRS module may be disposed or stacked on top of the TCM cabinet 108 to utilize the height of the room provided with such a medicine dispensing system 100.

Alternatively, the medicine dispensing system 100 may also be used for dispensing western medicine, in which medicine in pill or capsule form or even powder form may be stored in different drawers or compartments of the cabinet.

Preferably, the system 100 further comprises an indicator module arranged to provide a visual indication to a dispenser indicating a physical location of each of the at least one prescription item. For example, the indicator module may comprise a plurality of indicators, such as LED 112, each provided on a compartment 106 or a drawer of the cabinet 108, which may light up to indicate that prescription items should be withdrawn from the associated drawer in a medication dispensing process. In addition, an e-ink label/tag 114 may be provided to indicate names of prescription items stored in the drawer of the cabinet 108, which allow the herbalist or the medication dispensers to easily verify a correct prescription item to be withdrawn from the drawer.

Alternatively, other types or forms of visual indication may be provided to facilitate medication dispensing. For example, by flashing the labels/tags 114 to indicate that a particular prescription item is stored in the tagged drawer. In an alternative example, automatically opening the drawer, e.g. by push it out from the cabinet with a small gap, may indicate that prescription item should be withdrawn from the drawer.

Yet in an alternative example, the e-ink label/tag 114 and indicators 112 may be deployed on multiple separate bottles of Chinese medicine which may be in powder form, and the medication dispenser may easily locate the required prescription item in powder form easily by locating the lighting up LEDs.

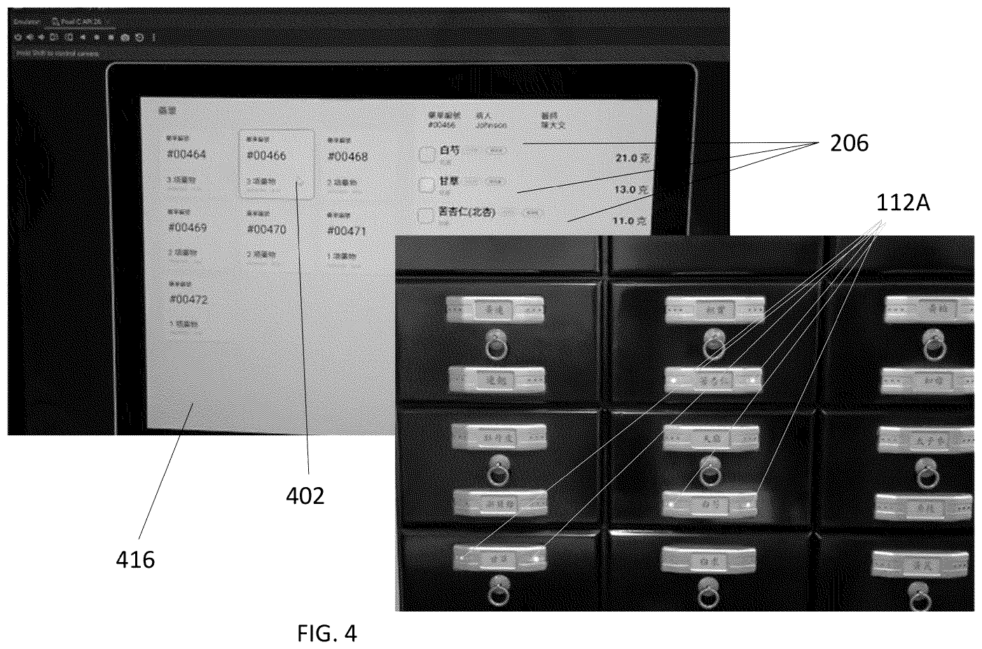

With reference to FIG. 4, once the prescription is received by the dispenser control module 102, the prescription is shown on the display module 116 or tablet computer 416, and a medicine dispenser may manually choose the prescription 402 to follow up, i.e. collect all the required prescription items 206 from the TCM cabinet 108 and dispense the medicine to the patient. For example, prescription #00466, including prescription items of 21.0 g White Peony Root (Baishao), 13.0 g Liquorice Root (Gancao) and 11.0 g Ansu Apricot Seed (Beixingren) may be chosen.

Upon prescription #00466 being selected on the screen, a pair of first LEDs 112A, which may be green LEDs, positioned on both sides of each of the e-ink label/tag 114 displaying the prescription items Baishao, Gancao and Beixingren may light up, indicating that the positions of the drawers storing these prescription items. An herbalist or pharmacist may withdraw these prescription items 206 of the medication prescription 402 from the cabinet 108 accordingly. In one example embodiment, the LED indicators may be deactivated if the same prescription on the screen is selected again (or become unselected).

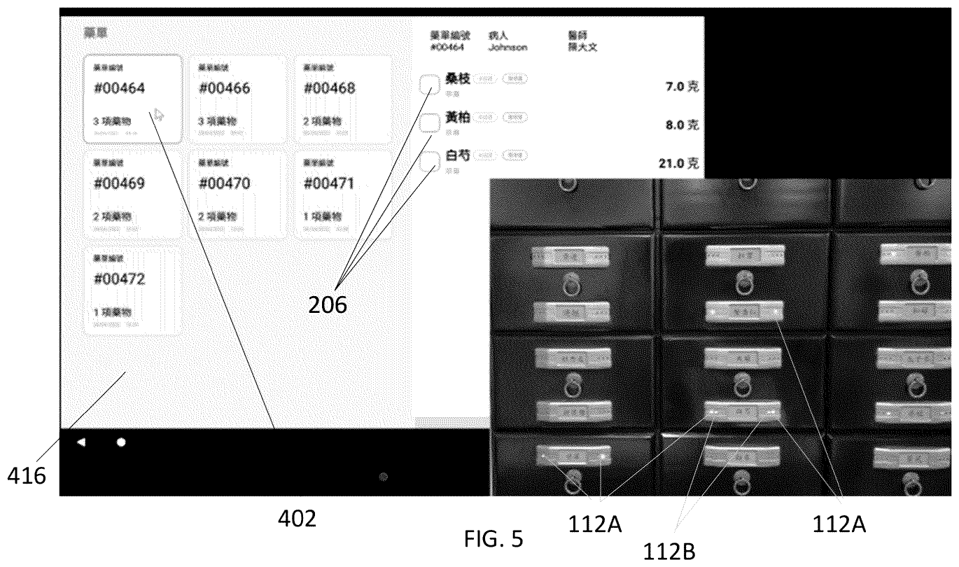

In addition, with reference to FIG. 5, the system 100 may preferably support dispensing multiple prescription handled by the same or different herbalist or pharmacist. In this example, when a second prescription #00464 is selected, the associated LED indicators 112 on the e-ink label/tag 114 displaying the listed prescription items light up to indicated the positions of these prescription items 206.

Referring to the Figure, the prescription items 206 include 7.0 g Mulberry Twig

(Sangzhi), 8.0 g Amur Corktree Bark (Huangbai) and 21.0 g White Peony Root (Baishao), and since the green LED 112A has been assigned to indicate the positions of the prescription items in the previously selected prescription #00466, a pair of second LEDs 112B, which may be blue LEDs, positioned on both sides of each of the e-ink label/tag 114 displaying the prescription items Sangzhi, Huangbai and Baishao light up to indicated the positions of these prescription items of prescription #00466. Similarly, the blue LEDs 112B may be deactivated if the same prescription #00466 on the screen is selected again (or become unselected).

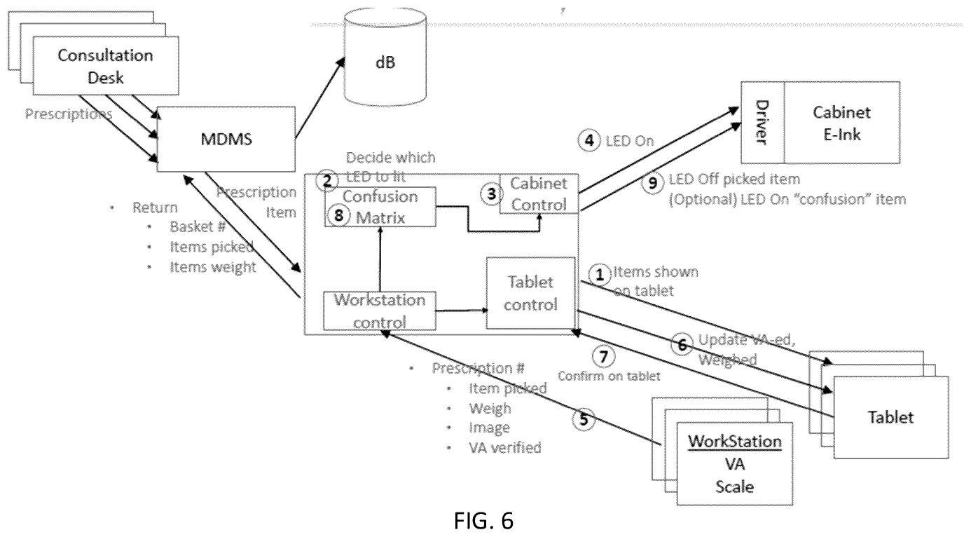

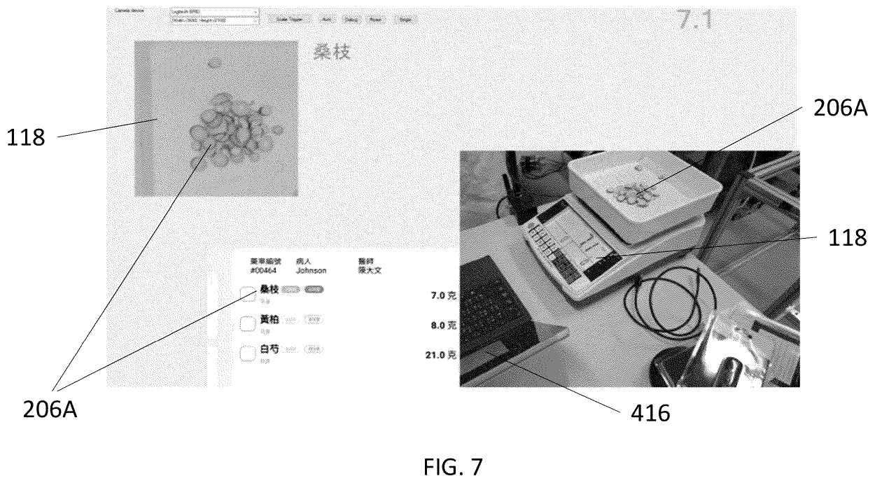

Advantageously, by providing multiple sets of LEDs 112 of different color or flashing modes, throughput of the medicine prescription dispensing process may be enhanced, whilst preventing prescription items of different prescription being dispensed. With reference to a flow diagram in FIG. 6 as well as the images/screenshots in FIGS. 7 to 10, an example of dispensing the medication prescription in FIG. 5 is further illustrated and described as follows. The process may begin by receiving the prescription prepared by a consultant, e.g. at a consultation desk, and then the prescription is processed by the medicine dispensing system 100 such that an herbalist may select the prescription item 206 displayed on the screen 416, e.g. prescription #00466 as shown in FIG. 5. The cabinet 108 control module 104, after assessing the TCM database 110 storing the positions of the appropriate drawers storing each of the prescription items required, may instruct the indicator module to light up the corresponding LEDs 112. Then the herbalist may start collecting the prescription items, one by one, from the drawers with activated LEDs 112.

The medicine dispensing system 100 also comprises an electronic weight 118 and an optical imager 120 or a camera for verifying the correct prescription item 206 being chosen and weighed, as well as a dispenser input module 122 and the display module 116 for displaying messages and receiving user input in different steps involved in the medication dispensing process. For example, the dispenser input module 122 and the display module 116 may be implemented as a computer or tablet computer 416 in communication with the dispenser control module 102.

With reference to FIG. 7, 7.1 g of Mulberry Twig (Sangzhi) 206A is weighed by the electronic weight 118 and the optical imager 120 may capture images of the prescription items 206 being weighted. The tablet 416 may notify the herbalist or pharmacist that the correct prescription item has been weighed and verified according to image recognition of the items being weighted. The herbalist may further provide a manual confirmation to the system 100, indicating that the prescription item has been weighed and dispensed, using the tablet computer 416, e.g. by checking a box or clicking a button next to the item Sangzhi 206A on the list.

In this example, the dispenser control module 102 is further arranged to update the visual indication provided to the dispenser indicating the physical locations of prescription items which have not been weighed and dispensed. Alternatively, the system 100 may consider that the prescription item has been collected if the drawer containing the prescription item has returned to its original position in the cabinet 108, and update the visual indication accordingly. Preferably, LEDs 112 on drawers containing prescription items which have not been weighed and dispensed are lighted up, all at once or one after one, and drawers containing prescription items which have been weighed or should not be collected remains deactivated.

Preferably, the display module 116 may also notify the dispenser an error or a potential error associated with the at least one prescription item 206 of the medication prescription. Example error may include the at least one prescription item being overweighed or incorrected dispensed.

Excessive use of traditional Chinese medicine, like western medicine, can have negative effects on the body. The risks of excessive use of traditional Chinese medicine are varied and can include liver and kidney damage, poisoning, allergic reactions, and drug interactions.

Liver and kidney damage is a common risk associated with the excessive use of traditional Chinese medicine. Some traditional Chinese medicines, when used excessively, can cause damage to the liver and kidneys, especially with long-term or improper use. This is because traditional Chinese medicines have active compounds that can interact with other medications, leading to liver and kidney damage.

Poisoning is another significant risk associated with the excessive use of traditional Chinese medicine. Some traditional Chinese medicines contain toxic ingredients that can lead to poisoning when used excessively. This risk is particularly high in the case of toxic traditional Chinese medicines.

Allergic reactions are another risk associated with the excessive use of traditional Chinese medicine. Some people may be sensitive to certain traditional Chinese medicines and develop allergic reactions that can cause skin redness, rashes, and shortness of breath.

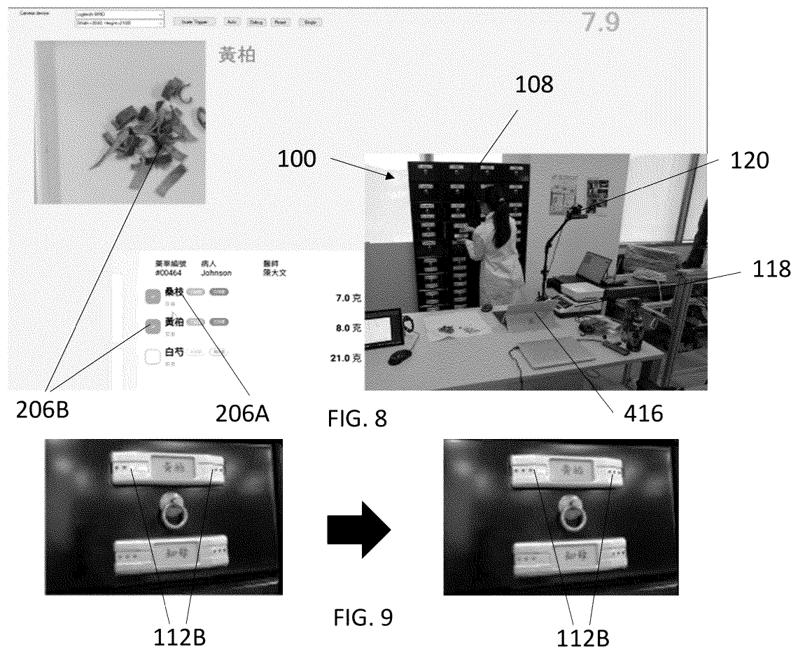

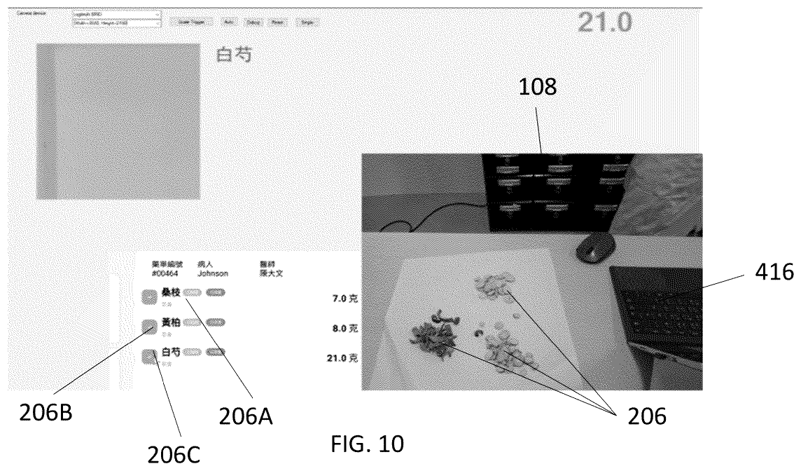

After receiving a manual confirmation that the prescription item Sangzhi 206A has been correctly withdrawn/collected, the herbalist may close/put back the drawer containing Sangzhi 206A and move on to withdraw the next prescription item, 8.0 g Amur Corktree Bark (Huangbai) 206B, by locating the blue LEDs on the cabinet 108 again. Referring to FIG. 8, 7.9 g of Huangbai 206B is weighed and verified by the image recognition, and referring to FIG. 9, once the manual confirmation is received by the system 100 that Huangbai 206B has been weighed and dispensed, the blue LEDs next to the e-ink tag 114 are deactivated. Lastly, by repeating the same procedures for the last prescription item White Peony Root (Baishao) 206C, as shown in FIG. 10, all three prescription items 206 are collected and ready for dispensing to the patient, e.g. returning back to the consultation desk or at the medication dispensing window.

In an alternative example, a potential error may include a wrong prescription item being selected due to confusion. For example, a warning message may be provided to the dispenser, e.g. via the display or tablet screen, to pay special attention to the prescription items to be withdrawn from the cabinet, in case confusion may occur.

Optionally or additionally, the medicine dispensing system 100 may further comprise a confusion matrix module 124, which may help preventing medication dispensing errors, such as by generating the visual indication to the dispenser indicating physical locations of a plurality of confusing prescription items in a staggered manner, i.e. not at the same time.

As described earlier, the use of Chinese herbal medicine also has its risks, and improper use may lead to some side effects and adverse reactions. One common problem is the confusion of Chinese herbal medicines, especially some Chinese herbal medicines that have similar appearance and properties, which can affect the efficacy of traditional Chinese medicine and cause adverse reactions.

For example, south apricot (Nanxing) and north apricot (Beixing) are two varieties of apricot, also known as sweet apricot and bitter apricot, respectively. Although they are both apricots, there are significant differences in taste, properties, and efficacy between south apricot and north apricot. South apricot is sweet in taste, warm in property, and can moisturize the lungs, stop cough, stimulate appetite and invigorate the spleen. Therefore, south apricot is widely used to treat diseases such as lung heat cough, phlegm and asthma, hypoxia, and poor appetite. On the other hand, north apricot is bitter in taste, cold in property, and can clear heat, detoxify, moisturize the intestines, and relieve constipation. Therefore, north apricot is widely used to treat diseases such as fever, thirst, phlegm and cough, constipation, as well as indigestion, dry mouth, and tongue dryness.

However, the difference in taste and properties between south apricot and north apricot also results in differences in their usage. Confusing south apricot and north apricot may lead to adverse reactions, so it is important to distinguish them when using them. In general, south apricot and north apricot should not be mixed or taken simultaneously. Additionally, the dosage of south apricot and north apricot should be adjusted according to the individual differences and the severity of the disease.

Here are some alternative examples of Chinese herbal medicines that may be easily confused:

Chuanxiong and Danggui: Chuanxiong is warm in nature and can activate blood circulation and relieve menstrual disorders and other gynecological diseases; Danggui is warm in nature and can nourish blood and activate blood circulation, commonly used to treat anemia and menstrual disorders. Both have similar effects, but Chuanxiong focuses more on activating blood circulation and relieving menstrual disorders, while Danggui focuses more on nourishing blood and activating blood circulation, so adjustments should be made according to the patient's condition and individual differences when used.

Licorice and Gansui: Licorice is neutral in nature and sweet in taste, commonly used to relieve the bitterness of Chinese medicine formulas and enhance their efficacy;

Gansui is cold in nature and can help to clear the urinary tract, commonly used to treat edema, constipation and other symptoms. Both have obvious differences in nature, taste, and efficacy, so it is necessary to pay attention to the distinction when used.

Honghua and Danggui: Honghua is warm in nature and can activate blood circulation and relieve stasis, commonly used to treat dysmenorrhea, bruises, and other symptoms; Danggui can nourish blood and activate blood circulation, commonly used to treat anemia and menstrual disorders. Both have similar effects, but Honghua focuses more on activating blood circulation and relieving stasis, while Danggui focuses more on nourishing blood and activating blood circulation, so adjustments should be made according to the patient's condition and individual differences when used.

In addition, there are many other Chinese herbal medicines that are easily confused, such as Gouqi and Dihuang, Huangqin and Huanglian, Chaihu and Huangqin, etc. These Chinese herbal medicines have some differences in appearance, properties, and efficacy, but are easily confused. Therefore, it is necessary to be extra careful when using them and use them under the guidance of a professional physician.

In one example embodiment, if medication prescription includes both south apricot (Nanxing) and north apricot (Beixing), the confusion matrix module may identify a potential confusion may occur, then even though both Nanxing and Beixing are to be withdrawn from the cabinet, the indicator module may activate the LEDs indicating the positions of Nanxing, then activate the LEDs indicating the positions of Beixing only after Nanxing has been correctly dispensed and verified, with the LEDs indicating the positions of Nanxing being deactivated.

In an alternative operation procedure, the medication dispensing system, also referred as a manual-herbal-dispensing system or TCM cabinet system, may operate as follows: firstly, the tablet control module may send prescription items to the tablet for display; then the confusion matrix module decides precedence of LED light on, e.g. Nanxing & Beixing should not lit up at the same time for the same prescription, then the cabinet control module 104 directs a cabinet driver to light up prescription items.

After the cabinet 108 LEDs light up accordingly, the herbalist or a TCM picker picks item from cabinet 108, weigh on the electronic scale and press scale button, then the tablet control update tablet with “weighed”, “verified”, and potentially display overweight/overdose, incorrect prescription item if necessary.

Once the herbalist confirmed with the picked item, the tablet control may receive the manual confirmation from the tablet, then the confusion matrix checks to see if further items need lit up, e.g. if Nanxing is confirmed picked, then the LED indicating the position of Beixing can light up, the cabinet control also turn OFF LED (optionally turn ON further LED) accordingly. By repeating the steps until all items picked, the system may return to an idle state until the next process when a new prescription is received or a next prescription is selected.

These embodiments may be advantageous in that, the medicine dispensing system help minimizing the chance of mistaken TCM prescription dispensing by guiding the dispenser to follow a step-by-step approach. In addition, errors may be avoided by utilizing the verification mechanism provided by the system to improve the quality of

TCM consultation services.

Although not required, the embodiments described with reference to the figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilized. This will include tablet computers, wearable devices, smart phones, Internet of Things (IOT) devices, edge computing devices, stand alone computers, network computers, cloud-based computing devices and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A medicine dispensing system comprising:
a dispenser control module arranged to receive a medication prescription including at least one prescription item;
a cabinet control module arranged to locate a position of each of the at least one prescription item stored in one of a plurality of predetermined compartment of a cabinet; and
an indicator module arranged to provide a visual indication to a dispenser indicating a physical location of each of the at least one prescription item.

2. The medicine dispensing system of claim 1, further comprising an electronic weight arranged to weigh the prescription item according to the medication prescription.

3. The medicine dispensing system of claim 2, further comprising an optical imager arranged to identify the prescription item being weighed.

4. The medicine dispensing system of claim 2, further comprising a dispenser input module arranged to receive a manual confirmation indicating that the prescription item has been weighed and dispensed.

5. The medicine dispensing system of claim 4, where the dispenser control module is further arranged to update the visual indication provided to the dispenser indicating the physical locations of prescription items which have not been weighed and dispensed.

6. The medicine dispensing system of claim 5, wherein the indicator module comprises a plurality of indicators each provided on a compartment of the cabinet, and wherein the physical locations of prescription items which have not been weighed and dispensed are indicated by lighting up an indicator provided on the respective compartment of the cabinet.

7. The medicine dispensing system of claim 1, further comprising a display module arranged to notify the dispenser an error or a potential error associated with the at least one prescription item of the medication prescription.

8. The medicine dispensing system of claim 7, wherein the error includes the at least one prescription item being overweighed or incorrected dispensed.

9. The medicine dispensing system of claim 7, wherein the potential error includes a wrong prescription item being selected due to confusion.

10. The medicine dispensing system of claim 9, further comprising a confusion matrix module arranged to generate the visual indication to the dispenser indicating physical locations of a plurality of confusing prescription items in a staggered manner.

11. A method for facilitating medicine dispensing, comprising the step of:
receiving a medication prescription including at least one prescription item;
locating a position of each of the at least one prescription item stored in one of a plurality of predetermined compartment of a cabinet; and
providing a visual indication to a dispenser indicating a physical location of each of the at least one prescription item.

12. The method of claim 11, further comprising the step of weighing the prescription item according to the medication prescription.

13. The method of claim 12, further comprising the step of identifying, by an optical imager, the prescription item being weighed.

14. The method of claim 12, further comprising the step of receiving a manual confirmation indicating that the prescription item has been weighed and dispensed.

15. The method of claim 14, further comprising the steps of updating the visual indication provided to the dispenser indicating the physical locations of prescription items which have not been weighed and dispensed.

16. The method of claim 15, wherein the physical locations of prescription items which have not been weighed and dispensed are indicated by lighting up an indicated provided on the respective compartment of the cabinet.

17. The method of claim 11, further comprising the step of notifying the dispenser an error or a potential error associated with the at least one prescription item of the medication prescription.

18. The method of claim 17, wherein the error includes the at least one prescription item being overweighed or incorrected dispensed.

19. The method of claim 17, wherein the potential error includes a wrong prescription item being selected due to confusion.

20. The method of claim 19, further comprising the step of providing the visual indication to the dispenser indicating physical locations of a plurality of confusing prescription items in a staggered manner.
